(19) United States
(12) Patent Application Publication
(10) Pub. No.: US 2024/0099905 A1
(43) Pub. Date: Mar. 28, 2024

(54) Sanitary Pad Device

(71) Applicant: Charmaine Magee, Gonzales, LA (US)

(72) Inventor: Charmaine Magee, Gonzales, LA (US)

(21) Appl. No.: 18/175,620

(22) Filed: Feb. 28, 2023

Related U.S. Application Data
(60) Provisional application No. 63/409,272, filed on Sep. 23, 2022.

(51) Int. Cl. A61F 13/472; A61F 13/56

(52) U.S. Cl. CPC A61F 13/47254; A61F 13/5616; A61F 2013/8497

(57) ABSTRACT

The present invention relates to a novel sanitary pad device. The device is a sanitary napkin that is designed to help prevent leakage of menstrual blood onto a woman's clothes. The sanitary pad device comprises a body component comprised of a pair of wing components on either side and an auxiliary rear component, which may be attached to the rear of the base component. The auxiliary rear component comprises a flap-like feature that extends from the rear of the base component via the user engaging a pull string to extend the auxiliary rear component. The auxiliary rear component is adapted for insertion between the thighs and buttocks of a user so as to contact at least a portion of the perineal region and extend toward the coccyx.

### CROSS-REFERENCE TO RELATED APPLICATION

The present application claims priority to, and the benefit of, U.S. Provisional Application No. 63/409,272, which was filed on Sep. 23, 2022, and is incorporated herein by reference in its entirety.

### FIELD OF THE INVENTION

The present invention relates generally to the field of sanitary pad devices. More specifically, the present invention relates to a sanitary pad that provides women with more protection and assists with containing menstrual flow. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in sanitary pad devices. Generally, standard women's hygiene products, like sanitary pads, may not be effective enough to fully cover and absorb during a woman's menstrual cycle. Further, some women are not comfortable using a tampon and rely solely on sanitary pads. Traditional sanitary pads do not extend to the buttocks area where leakage commonly occurs, especially while sleeping or performing activities. When leakage occurs, clothing or bedding may become stained causing embarrassment and inconvenience.

Thus, for many women the use of available sanitary pads is accompanied by anxiety over their effectiveness, particularly so in some particular use contexts. For example, many women will avoid sleeping lying supine, or will do so worrying about the possibility of leakage. Further, even sitting in a more relaxed or reclined posture may, especially for those women suffering a heightened degree of discharge, raise the fear of marked clothing and embarrassment. Leakage past the rear end of conventional pads when the body is in one of the above-described positions is possible because the essentially flat pads cannot conform closely enough to the junction between the perineum, thighs, and buttocks to prevent passage of the menses through the crevice formed between them.

Accordingly, it is an object of the present invention to address or ameliorate some of the above disadvantages of current sanitary pads. Thus, the present invention relates to a sanitary pad device that includes a flap-like feature that extends from the back of the pad to the buttocks area to prevent leakage and maintain maximum absorption coverage.

Therefore, there exists a long-felt need in the art for a sanitary pad device that provides women with a more protective sanitary pad to assist with containing menstrual flow. There is also a long-felt need in the art for a sanitary pad device that features a flap-like feature that extends from the pad to the buttocks area to prevent leakage and maintain maximum absorption coverage. Further, there is a long-felt need in the art for a sanitary pad device that offers buttocks coverage not offered by standard sanitary pads or tampons. Moreover, there is a long-felt need in the art for a device that prevents leakage that may occur while sleeping on your back, onto your clothing or bedding. Further, there is a long-felt need in the art for a sanitary pad device that assists female special needs consumers with a more comfortable monthly menstrual cycle. Finally, there is a long-felt need in the art for a sanitary pad device that utilizes a super-absorbent polymer for absorption means.

The subject matter disclosed and claimed herein, in one embodiment thereof, comprises a sanitary pad device. The device is a sanitary napkin that is designed to help prevent leakage of menstrual blood through a woman's clothes. The sanitary pad device comprises a body component comprised of a pair of wing components on either side and an auxiliary rear component, which may be attached to the rear of the base component. The base component is constructed in the manner of a conventional sanitary pad for the absorption of menstrual discharge and includes an adhesive component on an underside of the base component. The auxiliary rear component comprises a flap-like feature that extends from the rear of the base component via a user engaging a pull string to extend the auxiliary rear component. The auxiliary rear component is adapted for insertion between the thighs and buttocks of a user so as to contact at least a portion of the perineal region or its extension rearward of the vagina and extend toward the coccyx. Further, the sanitary pad device is constructed of a supporting layer of material overlaid with soft absorbent padding layers, both being of similar material and consistency as commonly used in the construction of conventional sanitary pads.

In this manner, the sanitary pad device of the present invention accomplishes all of the foregoing objectives and provides users with a device that provides maximum comfort and protection for a user. The device includes a flap-like feature that extends from the pad to the buttocks area to prevent leakage. The device can be manufactured of a super-absorbent polymer that can vary in dimension and size.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a sanitary pad device. The device is a sanitary napkin that is designed to help prevent leakage of menstrual blood onto a woman's clothes. The sanitary pad device comprises a body component comprised of a pair of wing components on either side and an auxiliary rear component, which may be attached to the rear of the base component. The base component is constructed in the manner of a conventional sanitary pad for the absorption of menstrual discharge. The auxiliary rear component is adapted for insertion between the thighs and buttocks of a user, so as to contact at least a portion of the perineal region.

The problem of seepage of menstrual discharge or other bodily fluids from the rear end of a conventionally constructed, substantially flat sanitary pad, is experienced by many women and particularly, by those women suffering a heavier than average discharge, or that participate in various athletic activities and/or while sleeping during their menstrual cycle. Specifically, the rear end of a flat sanitary pad when in use, generally positions in the region of the lower portion of the buttocks. Especially when a user is in a supine position, or even when sitting in a relaxed position, seepage of the menses may pass into the crevice between the buttocks and escape past the rear end of the pad.

In one embodiment, the present invention provides an auxiliary rear component, which may be attached to the rear of a conventional flat sanitary pad, particularly in situations where the discharge is heavy and for use at night or during strenuous activities. As well as being attachable to a conventional sanitary pad, the present invention also encompasses alternative arrangements in which the auxiliary rear component is attached either to the underwear or the skin of a user.

In one embodiment, the sanitary pad device comprises a body component and an auxiliary rear component constructed in the manner of a conventional sanitary pad for the absorption of menstrual discharge. The body component can be any suitable size and shape as is known in the art, based on the shapes and sizes of conventional sanitary pads. The auxiliary rear component is typically shaped like a long, skinny triangle, a slender pie-shape or a wedge-shaped structure, or any other suitable shape as is known in the art. In one embodiment, the auxiliary rear component may be given a decorative shape, for example a heart shape or other suitable shape.

The present invention is not limited to pads having substantially planar auxiliary components. A non-planar auxiliary component, having a cross-sectional profile that is anatomical in shape, aids in providing a comfortable, intimate fit to the user's body when inserted in the user's intergluteal crevice.

In one embodiment, the wedge-shaped structure of the auxiliary rear component is sized and shaped for insertion between the thighs and buttocks of a user, so as to contact at least a portion of the perineal region or its extension rearward of the vagina and extend toward the coccyx. Thus, in use, the rear edge of the body component may be positioned considerably further toward the coccyx than is possible with conventional sanitary pads. Indeed, the outer end of the auxiliary rear component may project from between the buttocks, covering the region of the coccyx.

In one embodiment, the body component and the auxiliary rear component is constructed of a supporting layer of material overlaid with soft absorbent padding layers, both being of similar material and consistency as commonly used in the construction of sanitary pads, such as super-absorbent polymers. Generally, the body component and the auxiliary rear component are both flexible and resilient.

In one embodiment, the auxiliary rear component may be provided in a range of sizes to suit variations in the body sizes of users and variations in volume of menstrual discharge. Thus, the wedge-shaped auxiliary rear component may be made available in a range of heights, lengths, and thicknesses. Further, the auxiliary rear component and the body component may be provided in selected grades of absorbency.

In one embodiment, the body component and the auxiliary rear component comprise an absorbent core positioned between a pad cover material and a barrier layer. This body component comprises a front end and a rear end. In the preferred embodiment of the present invention, the absorbent core is of sufficient length to only cover the length of the user's labia, that is, it is in the range 8.0 cm to 13.1 cm in length. The length of the main body component is preferably greater than the length of the absorbent core, so that a perimeter of the barrier layer and cover material surrounds the absorbent core.

In one embodiment, the widest portion of the auxiliary rear component is adjacent to the main body component and the narrowest portion is at the distal end, ending in a rounded point. The widest portion has a width of 7 cm, but can vary with the width of the main body component, from 7 cm to 10 cm. The length of the auxiliary rear component extends 4 cm beyond the end of the main body component. Thus, the length of the auxiliary rear component can range from 3 cm to 7 cm.

In the preferred embodiment, the main body of the pad is adapted to be worn outside of and in close proximity to the vagina of a wearer. Accordingly, in this embodiment, the main pad body is substantially planar on its body-facing side. Additional embodiments, while also substantially planar, have some taper in a front to back direction, or in a side-to-side direction, or both.

The width of the main body component most preferably varies along the length, becoming narrower at the rear end of the main body component. It could be relatively constant in width as well. In the preferred embodiment with a variable width, the maximum width occurs near the front end. The width there is in the range of approximately 8 cm to 10 cm. In the preferred embodiment, the main body component is most narrow at the rear end near the auxiliary rear component to thereby provide a more comfortable fit. Further, in the preferred embodiment, the narrowest part of the main body component should approximately equal the width of the auxiliary rear component.

In another embodiment, the auxiliary rear component comprises a pull string or other suitable component which enables a user to extend the auxiliary rear component for use. Generally, the auxiliary rear component is folded up under the body component when not in use. A user then grasps the pull string and pulls the string to extend out the auxiliary rear component for use.

In one embodiment, the sanitary pad device, including the body component and the auxiliary rear component, comprises a generally flat underside prepared with an adhesive layer, which is protected prior to use by a peel-off strip. In at least one embodiment, an adhesive layer may form at least a border extending along the complete periphery of the underside of the body component with protective stripping. Alternatively, the whole of underside may be provided with an adhesive layer, or the underside may be provided with a number of adhesive areas, for example, in strips. As well as providing for adhesion to a sanitary pad, this arrangement allows for the auxiliary rear component to also be adhered to the inside of the user's underwear.

Accordingly, in use, the peel-off strip is removed from the underside of the body component and the auxiliary rear component of the device is applied to the underwear of a user. The adhesive layer is formulated to be of sufficient strength so that once applied, the auxiliary rear component cannot accidentally be dislodged from the body component or from the user's underwear. Further, the peel-off layer protecting any adhesive layer in this embodiment is made of a soft non-irritating material, so that where an adhesive layer is not required and the protective layer remains in place, it does not cause irritation to the skin of a user.

In one embodiment, the auxiliary rear component may be formed of the same absorbent padding layer material as the body component, and may be integral with it, or may be separately constructed but permanently affixed to the body component. Further, the auxiliary rear component may be adapted for attachment to a conventional flat sanitary pad by adhesion, either to the upper surface or to the underside of the sanitary pad. Specifically, the auxiliary rear component is adapted for attachment at the rear of a conventional sanitary pad to form an extension of the pad. Construction adhesives as well as heat are exemplary means to attach the auxiliary rear component to the main body component.

In another embodiment, the sanitary pad device comprises one or more wings extending from each lateral side of the main body component. These wings are used to further secure the sanitary pad device to the user's undergarments. Such wings are well known in the sanitary pad industry.

In use, the configuration of the auxiliary rear component of the present invention allows for ease of positioning the component for protection against rearward leakage. Because it is specifically designed for relatively deep insertion between the buttocks, it is retained in that position with little risk of slippage. Further, where desired, the adhesive layer of the auxiliary rear component allows accurate positioning and attachment to a user's underwear. The adhesive connection between the auxiliary rear component and the rear end of a conventional sanitary pad ensures that any fluid passing the rear end is safely transferred to the auxiliary rear component and absorbed.

Specifically, by being wedged between the thighs and buttocks and in contact with at least a portion of the perineal area or its extension rearward of the vagina towards the coccyx, the auxiliary rear component acts as a barrier for fluids leaking toward the back of the body, particularly when the body is supine for sleep, when in a reclined sitting position, or when the body is performing an activity.

It will be understood that the sanitary pad device may be applied for use in a number of ways. The sanitary pad device may firstly be applied directly to the body of a user or the sanitary pad device may be affixed inside underwear by means of the adhesive on the underside of the device.

In yet another embodiment, the sanitary pad device comprises a plurality of indicia.

In yet another embodiment, a method of preventing leakage of menstrual blood onto a women's clothes is disclosed. The method includes the steps of providing a sanitary pad device comprising a body component with an auxiliary rear component which is extended out for use via a pull string. The method also comprises removing the adhesive covering on an underside of the body component. Further, the method comprises securing the device within a user's underwear via positioning the adhesive component on the interior of the underwear. The method comprises pulling out the pull string to extend the auxiliary rear component. Finally, the method comprises securing the auxiliary rear component to the buttocks area of a user's underpants via the adhesive component.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one embodiment of the sanitary pad device of the present invention showing the top of the body component with the auxiliary rear component extended in accordance with the disclosed architecture;

FIG. 2 illustrates a front perspective view of one embodiment of the sanitary pad device of the present invention showing the bottom of the body component with the adhesive component in accordance with the disclosed architecture;

FIG. 3 illustrates a side perspective view of one embodiment of the sanitary pad device of the present invention showing the device in position in accordance with the disclosed architecture;

FIG. 4 illustrates a front perspective view of one embodiment of the sanitary pad device of the present invention showing the device in use between the buttocks area in accordance with the disclosed architecture; and

FIG. 5 illustrates a flowchart showing the method of preventing leakage of menstrual blood through a women's clothes in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a sanitary pad device that provides women with a more protective sanitary pad to assist with containing menstrual flow. There is also a long-felt need in the art for a sanitary pad device that features a flap-like feature that extends from the pad to the buttocks area to prevent leakage and maintain maximum absorption coverage. Further, there is a long-felt need in the art for a sanitary pad device that offers buttocks coverage not offered by standard sanitary pads or tampons. Moreover, there is a long-felt need in the art for a device that prevents leakage that may occur while sleeping on your back, as well as leakage onto your clothing or bedding. Further, there is a long-felt need in the art for a sanitary pad device that assists female special needs consumers with a more comfortable monthly menstrual cycle. Finally, there is a long-felt need in the art for a sanitary pad device that utilizes a super-absorbent polymer for absorption means.

The present invention, in one exemplary embodiment, is a novel sanitary pad device. The device is a sanitary napkin that is designed to help prevent leakage of menstrual blood onto a woman's clothes. The sanitary pad device comprises a body component comprised of a pair of wing components on either side and an auxiliary rear component, which may be attached to the rear of the base component. The base component is constructed in the manner of a conventional sanitary pad for the absorption of menstrual discharge and includes an adhesive component on an underside of the base component. The auxiliary rear component comprises a flap-like feature that extends from the rear of the base component via a user engaging a pull string to extend the auxiliary rear component. The auxiliary rear component is adapted for insertion between the thighs and buttocks of a user so as to contact at least a portion of the perineal region.

The present invention also includes a novel method of preventing leakage of menstrual blood onto a women's clothes. The method includes the steps of providing a sanitary pad device comprising a body component with an auxiliary rear component, which is extended out for use via a pull string. The method also comprises removing the adhesive covering on an underside of the body component. Further, the method comprises securing the device within a user's underwear via positioning the adhesive component on the interior of the underwear. The method comprises pulling out the pull string to extend the auxiliary rear component. Finally, the method comprises securing the auxiliary rear component to the buttocks area of a user's underpants via the adhesive component.

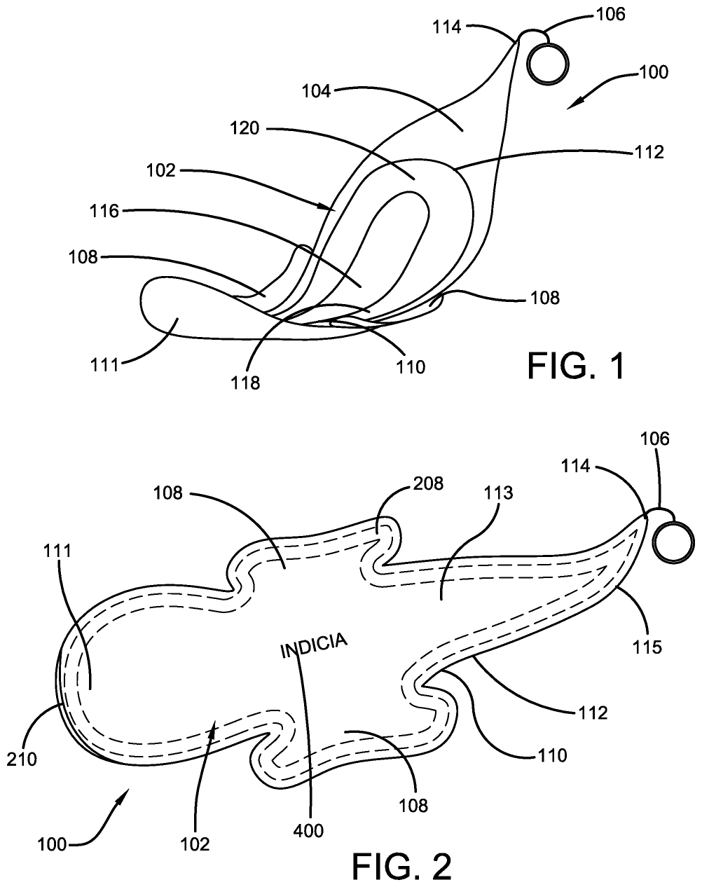

Referring initially to the drawings, FIG. 1 illustrates a perspective view of one embodiment of the sanitary pad device 100 of the present invention. In the present embodiment, the sanitary pad device 100 is an improved sanitary pad device 100 that prevents leakage of menstrual blood onto a woman's clothes. Specifically, the sanitary pad device 100 comprises a body component 102 with an auxiliary rear component 104 that is extended out via a pull string 106 and a pair of wing components 108 on either side 110 of the body component 102. The body component 102 is constructed in the manner of a conventional sanitary pad for the absorption of menstrual discharge. The auxiliary rear component 104 is adapted for insertion between the thighs 200 and buttocks 202 of a user 204, so as to contact at least a portion of the perineal region.

The problem of seepage of menstrual discharge or other bodily fluids from the rear end of a conventionally constructed, substantially flat sanitary pad, is experienced by many women and particularly by those women suffering a heavier than average discharge, that participate in various athletic activities, and/or while sleeping during their menstrual cycle. Specifically, the rear end of a flat sanitary pad when in use, generally positions in the region of the lower portion of the buttocks 202. When a user 204 is in a supine position, or even when sitting in a relaxed position, seepage of the menses may pass into the crevice between the buttocks 202 and escape past the rear end of the pad.

Furthermore, the present invention provides an auxiliary rear component 104, which may be attached to the rear 112 of a conventional flat sanitary pad, particularly in situations where the discharge is heavy and for use at night or during strenuous activities. As well as being attachable to a conventional sanitary pad, the present invention also encompasses alternative arrangements in which the auxiliary rear component 104 is attached either to the underwear or the skin of a user 204.

In one embodiment, the sanitary pad device 100 comprises a body component 102 and an auxiliary rear component 104 constructed in the manner of a conventional sanitary pad for the absorption of menstrual discharge. The body component 102 can be any suitable size and shape as is known in the art, based on the shapes and sizes of conventional sanitary pads. The auxiliary rear component 104 is typically shaped like a long, skinny triangle, a slender pie-shape or a wedge-shaped structure, or any other suitable shape as is known in the art. In one embodiment, the auxiliary rear component 104 may be given a decorative shape, for example a heart shape or other suitable shape as is known in the art.

The present invention is not limited to sanitary pads having substantially planar auxiliary components. In one embodiment, a non-planar auxiliary rear component 206 is utilized, having a cross-sectional profile that is anatomical in shape, which aids in providing a comfortable, intimate fit to the user's body when inserted in the user's intergluteal crevice (as shown in FIG. 2).

Further, the wedge-shaped structure of the auxiliary rear component 104 is sized and shaped for insertion between the thighs 200 and buttocks 202 of a user 204, so as to contact at least a portion of the perineal region or its extension rearward of the vagina and extending toward the coccyx. Thus, in use, the rear edge 112 of the body component 102 may be positioned considerably further toward the coccyx than is possible with conventional sanitary pads. Indeed, the outer end 114 of the auxiliary rear component 104 may project from between the buttocks 202 covering the region of the coccyx.

Furthermore, the body component 102 and the auxiliary rear component 104 is constructed of a supporting layer of material overlaid with soft absorbent padding layers, both being of similar material and consistency as commonly used in the construction of sanitary pads, for example, such as super-absorbent polymers. Generally, the body component 102 and the auxiliary rear component 104 are both flexible and resilient.

Additionally, the auxiliary rear component 104 may be provided in a range of sizes to suit variations in the body sizes of users 204 and variations in volume of menstrual discharge. Thus, the wedge-shaped auxiliary rear component 104 may be made available in a range of heights, lengths, and thicknesses. Further, the auxiliary rear component 104 and the body component 102 may be provided in selected grades of absorbency.

In one embodiment, the body component 102 and the auxiliary rear component 104 comprise an absorbent core 116 positioned between a pad cover material 118 and a barrier layer 120. The body component 102 comprises a front end 111 and a rear end 112. In the preferred embodiment of the present invention, the absorbent core 116 is of sufficient length to only cover the length of the user's labia, that is, it is in the range 8.0 cm to 13.1 cm in length. The length of the main body component 102 is preferably greater than the length of the absorbent core 116, so that a perimeter of the barrier layer 120 and cover material 118 surrounds the absorbent core 116.

Further, the widest portion 113 of the auxiliary rear component 104 is adjacent to the main body component 102 and the narrowest portion 115 is at the distal end 114, ending in a rounded point. The widest portion 113 has a width of 7 cm, but can vary with the width of the main body component 102 from 7 cm to 10 cm. The length of the auxiliary rear component 104 extends 4 cm beyond the end 112 of the main body component 102. Thus, the length of the auxiliary rear component 104 can range from 3 cm to 7 cm.

In the preferred embodiment the main body component 102 of the pad device 100 is adapted to be worn outside of and in close proximity to the vagina of a user 204. Accordingly, in this embodiment the main pad body component 102 is substantially planar on its body-facing side. Additional embodiments, while also substantially planar, have some taper in a front to back direction, or in a side-to-side direction, or both.

The width of the main body component 102 most preferably varies along the length, becoming narrower at the rear end 112 of the main body component 102. It could be relatively constant in width as well. In the preferred embodiment with a variable width, the maximum width occurs near the front end 111. The width there is in the range of approximately 8 cm to 10 cm. In the preferred embodiment, the main body component 102 is most narrow at the rear end 112 near the auxiliary rear component 104 to thereby provide a more comfortable fit. Further, in the preferred embodiment the narrowest part of the main body component 102 should approximately equal the width of the auxiliary rear component 104.

In one embodiment, the auxiliary rear component 104 comprises a pull string 106 or other suitable component which enables a user 204 to extend the auxiliary rear component 104 for use. Generally, the auxiliary rear component 104 is folded up under the body component 102 when not in use. A user 204 then grasps the pull string 106 and pulls the string to extend out the auxiliary rear component 104 for use.

In another embodiment, the sanitary pad device 100 comprises one or more wing components 108 extending from each lateral side 110 of the main body component 102. These wing components 108 are used to further secure the sanitary pad device 100 to the user's undergarments. Such wing components 108 are well known in the sanitary pad industry.

As shown in FIG. 2, the sanitary pad device 100, including the body component 102 and the auxiliary rear component 104, comprises a generally flat underside prepared with an adhesive layer 208, which is protected prior to use by a peel-off strip 210. In at least one embodiment, an adhesive layer 208 may form at least a border extending along the complete periphery of the underside of the body component 102 with protective stripping 210. Alternatively, the whole of underside may be provided with an adhesive layer 208, or the underside may be provided with a number of adhesive areas 208, for example, in strips. As well as providing for adhesion to a sanitary pad, this arrangement allows for the auxiliary rear component 104 to also be adhered to the inside of the user's underwear.

Accordingly, in use, the peel-off strip 210 is removed from the underside of the body component 102 and the auxiliary rear component 104 and the device 100 is applied to the underwear of a user 204. The adhesive layer 208 is formulated to be of sufficient strength so that once applied, the auxiliary rear component 104 cannot accidentally be dislodged from the body component 102 or from the user's underwear. Further, the peel-off layer 210 protecting any adhesive layer 208 in this embodiment is made of a soft non-irritating material, so that where an adhesive layer 208 is not required and the protective layer 210 remains in place, it does not cause irritation to the skin of a user 204.

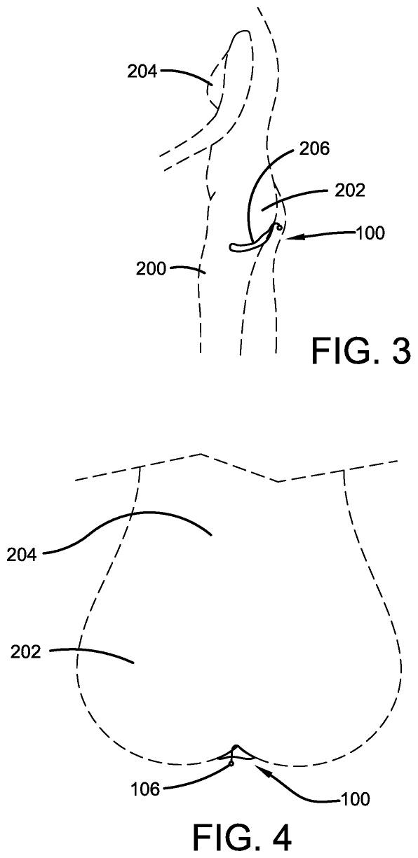

As shown in FIG. 3, the auxiliary rear component 104 may be formed of the same absorbent padding layer material as the body component 102, and may be integral with it, or may be separately constructed but permanently affixed to the body component 102. Further, the auxiliary rear component 104 may be adapted for attachment to a conventional flat sanitary pad by adhesion either to the upper surface or to the underside of the sanitary pad. Specifically, the auxiliary rear component 104 is adapted for attachment at the rear 112 of a conventional sanitary pad to form an extension of the pad. Construction adhesives as well as heat are exemplary means to attach the auxiliary rear component 104 to the main body component 102.

As shown in FIG. 4, in use, the configuration of the auxiliary rear component 104 of the present invention allows for ease of positioning the component for protection against rearward leakage. Because it is specifically designed for relatively deep insertion between the buttocks 202, it is retained in that position with little risk of slippage. Further, where desired, the adhesive layer 208 of the auxiliary rear component 104 allows accurate positioning and attachment to a user's underwear. The adhesive connection between the auxiliary rear component 104 and the rear end 112 of a conventional sanitary pad ensures that any fluid passing the rear end 112 is safely transferred to the auxiliary rear component 104 and absorbed.

Specifically, by being wedged between the thighs 200 and buttocks 202 and in contact with at least a portion of the perineal area or its extension rearward of the vagina toward the coccyx, the auxiliary rear component 104 acts as a barrier to fluids leaking towards the back of the body, particularly when the body is supine for sleep, when in a reclined sitting position, or when the body is performing an activity.

It will be understood that the sanitary pad device 100 may be applied for use in a number of ways. The sanitary pad device 100 may firstly be applied directly to the body of a user 204 or the sanitary pad device 100 may be affixed inside underwear by means of the adhesive layer 208 on the underside of the device 100.

Furthermore, the sanitary pad device 100 comprises a plurality of indicia 400. The body component 102 of the device 100 may include advertising, trademark, letters, designs, or characters, printed, painted, stamped, or integrated into the body component 102, or any other indicia 400 as is known in the art. Specifically, any suitable indicia 400 as is known in the art can be included, such as, but not limited to, patterns, logos, emblems, images, symbols, designs, letters, words, characters, animals, advertisements, brands, etc., that may or may not be female, menstrual, or brand related.

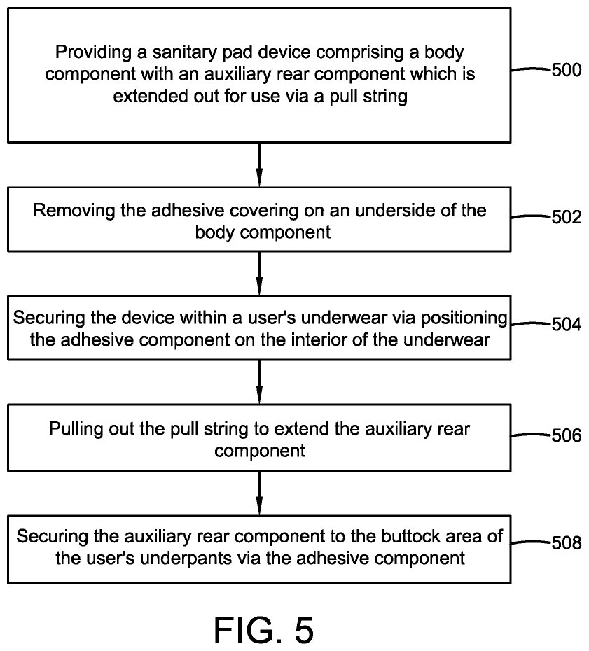

FIG. 5 illustrates a flowchart of the method of preventing leakage of menstrual blood onto a women's clothes. The method includes the steps of at 500, providing a sanitary pad device comprising a body component with an auxiliary rear component, which is extended out for use via a pull string. The method also comprises at 502, removing the adhesive covering on an underside of the body component. Further, the method comprises at 504, securing the device within a user's underwear via positioning the adhesive component on the interior of the underwear. The method comprises at 506, pulling out the pull string to extend the auxiliary rear component. Finally, the method comprises at 508, securing the auxiliary rear component to the buttocks area of a user's underpants via the adhesive component.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “sanitary pad device”, “pad device”, and “device” are interchangeable and refer to the sanitary pad device 100 of the present invention.

Notwithstanding the foregoing, the sanitary pad device 100 of the present invention can be of any suitable size and configuration as is known in the art without affecting the overall concept of the invention, provided that it accomplishes the above-stated objectives. One of ordinary skill in the art will appreciate that the sanitary pad device 100 as shown in FIGS. 1-5 are for illustrative purposes only, and that many other sizes and shapes of the sanitary pad device 100 are well within the scope of the present disclosure. Although the dimensions of the sanitary pad device 100 are important design parameters for user convenience, the sanitary pad device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sanitary pad device that prevents leakage of menstrual blood through a user's clothes during activities or sleeping, the sanitary pad device comprising:
a body component; and
an auxiliary rear component which can be extended out for use;
wherein the body component is secured within a user's underwear during use;
wherein the body component absorbs menstrual discharge; and
further wherein the auxiliary rear component is adapted for insertion between a thigh and buttocks of the user, so as to contact at least a portion of a user's perineal region.

2. The sanitary pad device of claim 1, wherein the body component is constructed in a manner of a conventional sanitary pad for absorption of menstrual discharge.

3. The sanitary pad device of claim 2, wherein the auxiliary rear component is attached to a rear of the body component.

4. The sanitary pad device of claim 3, wherein the auxiliary rear component is shaped in a wedge-shaped structure with a wider end secured to the body component and a narrower end positioned at a distal end from the body component.

5. The sanitary pad device of claim 3, wherein the auxiliary rear component is non-planar and anatomically shaped to fit a user's body.

6. The sanitary pad device of claim 1, wherein the body component and the auxiliary rear component comprise an absorbent core positioned between a pad cover material and a barrier layer.

7. The sanitary pad device of claim 6, wherein a perimeter of the barrier layer and the pad cover material surrounds the absorbent core.

8. The sanitary pad device of claim 7, wherein the auxiliary rear component comprises a pull string which enables the user to extend the auxiliary rear component for use.

9. The sanitary pad device of claim 8 further comprising at least one wing component extending from a lateral side of the body component.

10. The sanitary pad device of claim 9 further comprising a pair of wing components, one extending from each of the lateral sides of the body component.

11. The sanitary pad device of claim 10, wherein the body component and the auxiliary rear component comprise a generally flat underside with an adhesive layer.

12. The sanitary pad device of claim 11, wherein the adhesive layer is protected prior to use by a peel-off strip.

13. The sanitary pad device of claim 12, wherein the auxiliary rear component is integral with the body component and manufactured as one piece.

14. A sanitary pad device that prevents leakage of menstrual blood through a user's clothes during activities or sleeping, the sanitary pad device comprising:
a body component with a pair of wing components extending from each of lateral sides of the body component; and
an auxiliary rear component with a pull string which enables a user to extend the auxiliary rear component for use;
wherein the auxiliary rear component is attached to a rear of the body component;
wherein the body component and the auxiliary rear component comprise a generally flat underside with an adhesive layer for securing within a user's underwear during use;
wherein the body component and the auxiliary rear component comprise an absorbent core positioned between a pad cover material and a barrier layer for absorbing menstrual discharge;
wherein a perimeter of the barrier layer and the pad cover material surrounds the absorbent core; and
further wherein the auxiliary rear component is adapted for insertion between thigh and buttocks of the user, so as to contact at least a portion of a user's perineal region.

15. The sanitary pad device of claim 14 further comprising a plurality of indicia.

16. The sanitary pad device of claim 14, wherein the auxiliary rear component is integral with the body component and manufactured as one piece.

17. The sanitary pad device of claim 14, wherein the auxiliary rear component is shaped in a wedge-shaped structure with a wider end secured to the body component and a narrower end positioned at a distal end from the body component.

18. The sanitary pad device of claim 14, wherein the auxiliary rear component is non-planar and anatomically shaped to fit a user's body.

19. The sanitary pad device of claim 14, wherein the adhesive layer is protected prior to use by a peel-off strip.

20. A method of preventing leakage of menstrual blood through a user's clothes, the method comprising the following steps:
providing a sanitary pad device comprising a body component with an auxiliary rear component, which is extended out for use via a pull string;
removing the adhesive covering on an underside of the body component;
securing the device within a user's underwear via positioning the adhesive component on the interior of the underwear;
pulling out the pull string to extend the auxiliary rear component; and
securing the auxiliary rear component to the buttocks area of a user's underpants via the adhesive component.
